(19) United States
(12) Patent Application Publication
(10) Pub. No.: US 2004/0110963 A1
(43) Pub. Date: Jun. 10, 2004

(54) Novel hydrazones

(76) Inventors: Kaspar Burri, Binningen (CH); Johannes Hoffner, Ziefen (CH); Khalid Islam, Reinach (CH); Seema Mukhija, Ahornstrasse (CH)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/466,810

(22) PCT Filed: Jan. 18, 2002
(86) PCT No.: PCT/EP02/00474

(30) Foreign Application Priority Data
Jan. 22, 2001 (WO) PCT/EP01/00636

(51) Int. Cl. C07C 251/72; C07D 209/18

(52) U.S. Cl. 548/494; 548/537; 564/147

(57) ABSTRACT

The invention relates to novel hydrazone derivatives and their use as active ingredients in the preparation of pharmaceutical compositions. The invention also concerns related aspects including processes for the preparation of the compounds, pharmaceutical compositions containing one or more of those compounds and especially their use as anti-infectives.

The present invention relates to novel hydrazones of the general formula 1, to a process for the manufacture of these hydrazones, to pharmaceutical compositions containing them and to their use in the treatment of microbial infections.

Related hydrazones have been investigated previously, especially with regard to their potential as antitumor agents: see Antonini et al., J. Med. Chem. 1981, 24, 1181-1184. Notably PIH (Pyridoxal Isonicotinoyl Hydrazone) seem to display pronounced antiproliferative activity: Richardson, D. R.; Milnes, K. Blood 1997, 89, 3025-38. Moreover, azinyl and diazinyl hydrazones appear to act similarly: Easmon, J.; Heinisch, G.; Pürstinger, G.; Langer, T.; Osterreicher, J. K.; Grunicke, H. H.; Hofmann, J. J. Med. Chem., 1997, 40, 4420-4425. The inhibition of tumor growth seems to be linked to the iron (III) chelating property of PIH: Richardson, D. R. Antimicrob. Agents Chemother. 1997, 41, 2061-2063.

So far only peptides have been uncovered to inhibit the bacterial phosphotransferase system (PTS) which is a drug target system useful for identifying new anti-microbials. It has now been found that most of the hydrazones of formula 1 of the present invention are potent inhibitors of enzyme I of the bacterial phosphotransferase system (“PTS”) (compare table 1). Inhibition of enzyme I is expected to decrease bacterial virulence and pathogenicity, as demonstrated by gene knock-out studies (Eur. Pat. Appl. EP 0 866 075). Consequently, low molecular weight organic compounds affecting this phosphorylation cascade may be useful in the treatment of bacterial infections in human and/or veterinary medicine. It has also been found that a number of these compounds, that are active in PTS, exhibit antibacterial activity. Several compounds of formula 1 are very specific in exhibiting antibacterial activity consequently these compounds of formula 1 are generally useful to combat bacterial pathogens in human and animals, e.g. to combat Gram positive pathogens such as Staphylococcus aureus, Staphylococcus epidermidis, Enterococcus faecalis or Streptococcus pneumoniae etc., and Gram negatives like Haemophilus influenzae, Escherichia coli, Klebsiella pneumoniae or Proteus vulgaris.

The determination of activity of a compound of the present invention in the PTS may be summarized as follows:

Assay for Enzyme I Dependent PEP:

Peptide Phosphotransferase Activity.

[image]

To find inhibitors of Enzyme I of the PTS by high throughput screening, an in vitro assay based on spectrophotometric read out at 340 nm has been set up. The assay comprises of three major components, purified enzyme I in catalytic amounts, Phosphoenol Pyruvate (PEP) as the phosphoryl donor substrate and purified HPr as the phosphoryl acceptor substrate.

The assay couples the formation of pyruvate formed from PEP to lactate, catalyzed by lactate dehydrogenase. The disappearance of NADH, cofactor required by lactate dehydrogenase, is determined spectrophotometerically at 340 nm. The assay is done in a U-shaped microtiter plate format, and quantitation is done using microplate absorbance reader.

100 μl reaction mixture contained 0.8 mM PEP, 0.2 mM NADH, 3 μg lactate dehydrogenase (Boehringer Mannheim), 50 mM KPᵢ pH=7.5, 2.5 mM dithiothreitol, 2.5 mM NaF, 5 mM MgCI₂, and between 50 and 100 μM of the compound. The reaction is started by the addition of enzyme I (final concentration 0.75 μM). In a control experiment the compound is replaced by DMSO.

The results obtained are summarized table 1.

TABLE 1

| | | | Inhibition |
|---|---|---|---|
| | | | of PTS |
| | Exam- | Synthetic | (IC50 in |
| Compounds | ple | method | μM) |
| N′-(2,5-Dihydroxy-benzylidene)- | 1 | A | 15 |
| benzohydrazide | | | |
| N′-(2-Hydroxy-benzylidene)-2-(1H- | 2 | A | 50 |
| indol-3-yl)-acetohydrazide | | | |
| N′-(2,5-Dihydroxy-benzylidene)- | 3 | A | 6 |
| naphthalene-1-carbohydrazide | | | |
| 3,4,5-Trimethoxy-N′-(2,3,4-trihydroxy- | 4 | A | 15 |
| benzylidene)-benzohydrazide | | | |
| 2-Amino-5-chloro-N′-(2-hydroxy- | 5 | A | 6 |
| benzylidene)-benzohydrazide | | | |
| 3-Trifluoromethyl-N′-(2,4-dihydroxy- | 6 | A | 10 |
| benzylidene)-benzohydrazide | | | |
| 3-Methoxy-N′-[1-(2-hydroxy-phenyl)- | 7 | B | 8 |
| ethylidene]-benzohydrazide | | | |
| 3-Methoxy-N′-(2,5-dihydroxy- | 8 | A | 15 |
| benzylidene)-benzohydrazide | | | |
| 3,4-Dichloro-N′-(2,3,4-trihydroxy- | 9 | A | 75 |
| benzylidene)-benzohydrazide | | | |
| 4-Chloro-N′-(2,5-dihydroxy- | 10 | A | 8 |
| benzylidene)-benzohydrazide | | | |
| 4-Hydroxy-N′-(2,5-dihydroxy- | 11 | A | 0.5 |
| benzylidene)-benzohydrazide | | | |
| 3,4-Dichloro-N′-(2,5-dihydroxy- | 12 | A | 0.7 |
| benzylidene)-benzohydrazide | | | |
| 3-Chloro-N′-(2,5-dihydroxy- | 13 | A | 0.7 |
| benzylidene)-benzohydrazide | | | |
| 4-Hydroxy-3-methoxy-N′-(5-chloro-2- | 14 | A | 25 |
| hydroxy-benzylidene)-benzohydrazide | | | |
| N′-[1-(2,5-Dihydroxy-phenyl)- | 15 | A | 6 |
| ethylidene]-benzohydrazide | | | |
| N′-(2,5-Dihydroxy-benzylidene)-4- | 16 | A | 4 |
| hydroxy-3-methoxy-benzohydrazide | | | |
| N′-(2-Hydroxy-5-methyl-benzylidene)- | 17 | A | 6 |
| benzohydrazide | | | |
| 2-Methylamino-N′-(5-chloro-2-hydroxy- | 18 | A | 4 |
| benzylidene)-benzohydrazide | | | |
| 2-Methylamino-N′-(2,5-dihydroxy- | 19 | A | 2 |
| benzylidene)-benzohydrazide | | | |
| 3-Methyl-N′-(5-chloro-2-hydroxy- | 20 | A | 4 |
| benzylidene)-benzohydrazide | | | |
| 3-Trifluoromethyl-N′-(5-chloro-2- | 21 | A | 12 |
| hydroxy-benzylidene)-benzohydrazide | | | |
| 2-Methylamino-N′-[1-(2-hydroxy- | 22 | A | 2 |
| phenyl)-7ethylidene]-benzohydrazide | | | |
| N-[2-[1-(2-Benzoyl-hydrazono)-ethyl]- | 23 | A | 250 |
| phenyl]-acetamide | | | |
| 4-Chloro-N′-[1-(2-amino-phenyl)- | 24 | B | 0.8 |
| ethylidene]-benzohydrazide | | | |
| 3-Methoxy-N′-[1-(2-Amino-phenyl)- | 25 | B | 20 |
| ethylidene]-benzohydrazide | | | |
| N′-(2,3-Dihydroxy-benzylidene)- | 26 | A | 50 |
| benzohydrazide | | | |
| 3-Methoxy-N′-(2-hydroxy-benzylidene)- | 27 | A | 7 |
| benzohydrazide | | | |
| N′-(2,3,4-Trihydroxy-benzylidene)- | 28 | A | 3 |
| benzohydrazide | | | |
| N′-(2,4,5-Trihydroxy-benzylidene)- | 29 | A | 25 |
| benzohydrazide | | | |
| 3,4,5-Trimethoxy-N′-(2,4,5-trihydroxy- | 30 | A | 25 |
| benzylidene)-benzohydrazide | | | |
| 4-Bromo-N′-(2-hydroxy-benzylidene)- | 31 | A | 75 |
| benzohydrazide | | | |
| 3-Trifluoromethyl-N′-(2-hydroxy- | 32 | A | 7 |
| benzylidene)-benzohydrazide | | | |
| 3-Methyl-N′-(2,5-dihydroxy- | 33 | A | 2 |
| benzylidene)-benzohydrazide | | | |
| 3-Trifluoromethyl-N′-(2,5-dihydroxy- | 34 | A | 15 |
| benzylidene)-benzohydrazide | | | |
| 4-Hydroxy-N′-[1-(2,5-dihydroxy- | 35 | B | 1.75 |
| phenyl)-ethylidene]-benzohydrazide | | | |
| 4-chloro-N′-(2-hydroxy-3-chloro- | 36 | A | 100 |
| benzylidene)-benzohydrazide | | | |
| 4-Chloro-N′-(2,4-dihydroxy- | 37 | A | 20 |
| benzylidene)-benzohydrazide | | | |
| 3-Chloro-N′-(2-hydroxy-5-chloro- | 38 | A | 75 |
| benzylidene)-benzohydrazide | | | |

Biological Results

Antimicrobial susceptibility testing was performed in accordance with the National Committee for Clinical Laboratory Standards (NCCLS) procedure [M7-A5, 2001: Methods for Dilution Antimicrobial Susceptibility Tests for Bacteria That Grow Aerobically; Approved Standard-Fifth Edition American National Standard].

The results are obtained are summarized in table 2.

TABLE 2
In vitro Antibacterial Activity of Compounds
(Minimum Inhibitory Concentration (MIC) in micrograms/ml)

| | | | | Staphylococcus | |
|---|---|---|---|---|---|
| | | Synthetic | Escherichia | aureus | Staphylococcus |
| Name | example | method | coli DC2 | ATCC25923 | aureus 101 |
| N′-(2,5-Dihydroxy-benzylidene)- | 1 | A | 128 | 64 | nt |
| benzohydrazide | | | | | |
| 3,4,5-Trimethoxy-N′-(2,3,4-trihydroxy- | 4 | A | 128 | 128 | nt |
| benzylidene)-benzohydrazide | | | | | |
| 3-Trifluoromethyl-N′-(2,4-dihydroxy- | 6 | A | 32 | na | nt |
| benzylidene)-benzohydrazide | | | | | |
| 3,4-Dichloro-N′-(2,3,4-trihydroxy- | 9 | A | 32 | 8 | nt |
| benzylidene)-benzohydrazide | | | | | |
| 4-Chloro-N′-(2,5-dihydroxy- | 10 | A | na | 128 | nt |
| benzylidene)-benzohydrazide | | | | | |
| 4-Hydroxy-3-methoxy-N′-(5-chloro-2- | 14 | A | 128 | 128 | nt |
| hydroxy-benzylidene)-benzohydrazide | | | | | |
| 3-Trifluoromethyl-N′-(5-chloro-2- | 21 | A | na | 16 | nt |
| hydroxy-benzylidene)-benzohydrazide | | | | | |
| 4-Methoxy-N′-(2,3,4-trihydroxy- | 39 | A | 64 | 64 | 64 |
| benzylidene)-benzohydrazide | | | | | |
| 3,4-Dichloro-N′-(2,3-dihydroxy- | 40 | A | na | 4 | 4 |
| benzylidene)-benzohydrazide | | | | | |
| 3,5-Bis-(trifluoromethyl)-N′-(2,3,4- | 41 | A | na | 64 | 64 |
| trihydroxy-benzylidene)- | | | | | |
| benzohydrazide | | | | | |
| 3-Chloro-2-pyrrol-1-yl-N′-(2,3,4- | 42 | A | 128 | 32 | 32 |
| trihydroxy-benzylidene)- | | | | | |
| benzohydrazide | | | | | |
| 3-Chloro-2-pyrrol-1-yl-N′-(2-hydroxy- | 43 | A | na | 2 | 2 |
| 3,5-dichloro-benzylidene)- | | | | | |
| benzohydrazide | | | | | |
| 2-Pyrrol-1-yl-N′-(2,4,5-trihydroxy- | 44 | A | 128 | 64 | 64 |
| benzylidene)-benzohydrazide | | | | | |
| 4-Chloro-3-trifluoromethyl-N′-(2,3,4- | 45 | A | 2 | 0.5 | 1 |
| trihydroxy-benzylidene)- | | | | | |
| benzohydrazide | | | | | |
| 4-Chloro-3-trifluoromethyl-N′-(2- | 46 | A | na | 128 | 128 |
| hydroxy-3,5-dichloro-benzylidene)- | | | | | |
| benzohydrazide | | | | | |
| 4-Chloro-N′-(2,4,5-trihydroxy- | 47 | A | 64 | 8 | nt |
| benzylidene)-benzohydrazide | | | | | |
| N′-(2-Hydroxy-3,5-dichloro- | 48 | A | na | 128 | nt |
| benzylidene)-benzohydrazide | | | | | |
| 3-Chloro-N′-(2,3,4-trihydroxy- | 49 | A | 64 | 16 | nt |
| benzylidene)-benzohydrazide | | | | | |
| 3-Trifluoromethyl-N′-(2,4,5-trihydroxy- | 50 | A | na | 32 | nt |
| benzylidene)-benzohydrazide | | | | | |
| 3-Trifluoromethyl-N′-(2,3,4-trihydroxy- | 51 | A | 64 | 8 | nt |
| benzylidene)-benzohydrazide | | | | | |
| 3,4-Dichloro-N′-[1-(2,3,4-dihydroxy- | 52 | A | 64 | 4 | nt |
| phenyl)-ethylidene]-benzohydrazide | | | | | |
| 3,4-Dichloro-N-methyl-N′-(2,3,4- | 53 | A | na | 128 | nt |
| trihydroxy-benzylidene)- | | | | | |
| benzohydrazide | | | | | |

na means not active at concentrations less than 128 μg/ml
nt means not tested

The present invention relates to novel hydrazones of the general formula 1,

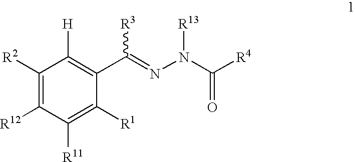

wherein R¹ represents lower alkyl-carbonylamino; formylamino; amino; hydroxy;

R² represents hydrogen; hydroxy; lower alkyl; fluoro; chloro;

R³ represents hydrogen; methyl; ethyl; isopropyl;

R¹¹ represents hydrogen; hydroxy; lower alkyl; lower alkoxy; fluoro; chloro; amino;

R¹² represents hydrogen; hydroxy; lower alkyl; lower alkoxy; fluoro; chloro; amino

R¹³ represents hydrogen; lower alkyl

R⁴ represents aryl; arylmethyl; indoyl methyl; mono-, di- or tri- substituted aryl, arylmethyl, which substituents may be lower alkyl, hydroxy, lower alkoxy, fluoro, chloro, bromo, trifluoromethyl, amino, lower alkylamino, lower alkylendioxy, N-pyrrolyl, 2-pyrrolyl, 3-pyrrolyl and which substituents may be the same or different;

in case R¹ represents amino and R², R¹¹, R¹², R¹³ and R³ represent hydrogen, R⁴ is not unsubstituted phenyl; phenylmethyl; 2-amino-phenyl; 2-hydroxy-phenyl; 4-chloro-phenyl;

in case R¹ represents amino and R², R¹¹, R¹² and R¹³ represent hydrogen and R³ represents methyl, R⁴ is not unsubstituted phenyl; 2-hydroxy-phenyl;

in case R¹ represents methyl-carbonylamino and R², R³, R¹¹, R¹³ and R¹² represent hydrogen, R⁴is not 4-hydroxy-3-methoxy-phenyl;

in case R¹ is hydroxy and R², R¹¹, R¹² and R¹³ represent hydrogen and R³ represents methyl, R⁴ is not unsubstituted phenyl; 4-methyl-phenyl; 2-methyl-phenyl; 2-hydroxy-phenyl; 4-methoxy-phenyl; 4-chloro-phenyl; 2-chloro-phenyl; 2,4,6-trimethyl-phenyl;

in case R¹ is hydroxy and R², R¹¹, R¹² and R¹³ represent hydrogen and R³ represents ethyl, R⁴ is not unsubstitued phenyl or 2-hydroxy-phenyl;

in case R¹ is hydroxy and R², R¹¹, R¹² and R³ represent hydrogen and R¹³ represents methyl, R⁴ is not unsubstituted phenyl;

in case R¹ is hydroxy and R², R¹¹, R¹², R¹³ and R³ represent hydrogen, R⁴ is phenyl substituted with 2-trifluoromethyl, 3-trifluoromethyl, 3-methoxy or (2-amino-5-chloro);

in case R¹ and R¹¹ represent hydroxy and R², R³, R¹² and R¹³ represent hydrogen, R⁴ is not 2-chloro-phenyl;

in case R¹ is hydroxy and R¹¹ is methoxy and R², R³, R¹² and R¹³ represent hydrogen, R⁴ is not unsubstituted phenyl; 2-hydroxy-phenyl; 2-chloro-phenyl; 4-hydroxy-3-methoxy-phenyl; 5-chloro-2-hydroxy-phenyl; 2-(3-hydroxy)-naphthyl; 2,4-dichloro-phenyl; 4-amino-3,5-dichloro-phenyl; 5-bromo-2-hydroxy-phenyl;

in case R¹, R¹¹ and R¹² represent hydroxy and R² and R¹³ represent hydrogen and R³ is methyl, R⁴ is not unsubstituted phenyl;

in case R¹ and R¹² represent hydroxy and R², R³, R¹¹ and R¹³ represent hydrogen, R⁴ is not unsubstituted phenyl; 2-hydroxy-phenyl; 4-methoxy-phenyl; 4-hydroxy-3-methoxy-phenyl; 2,4-dichloro-phenyl;

in case R¹ and R¹² represent hydroxy and R², R¹¹ and R¹³ represent hydrogen and R³ is methyl, R⁴is not unsubstituted phenyl; 2-hydroxy-phenyl;

in case R¹ is hydroxy and R¹² is methoxy and R², R³, R¹¹ and R¹³ represent hydrogen, R⁴ is not 4-hydroxy-3-methoxy-phenyl;

in case R¹ is hydroxy and R¹² is methoxy and R², R¹¹ and R¹³ represent hydrogen and R³ is methyl, R⁴ is not unsubstituted phenyl;

in case R¹ is hydroxy and R² is chloro and R³, R¹¹, R¹² and R¹³ represent hydrogen, R⁴ is not unsubstituted phenyl; 2-methyl-phenyl; 2-hydroxy-phenyl; 4-hydroxy-phenyl; 4-methoxy-phenyl; 4-chloro-phenyl; 5-chloro-2-hydroxy-phenyl; 2-hydroxy naphth-1-yl; 3-hydroxy naphth-2-yl; 2,4-dichloro-phenyl; 3,4-dichloro-phenyl; 3,4,5-trihydroxy-phenyl; 5-bromo-2-hydroxy-phenyl;

in case R¹ is hydroxy and R² and R¹¹ represent chloro and R³, R¹² and R¹³ represent hydrogen, R⁴ is not 2-hydroxy-phenyl; 5-chloro-2-hydroxy-phenyl; 3-hydroxy naphth-2-yl; 2-hydroxy-3,5-dichloro-phenyl; 5-bromo-2-hydroxy-phenyl; 3,5-dibromo-2-hydroxy-phenyl; N-pyrrolyl;

in case R¹ is hydroxy and R² and R³ represent methyl and R¹¹, R¹² and R¹³ represent hydrogen, R⁴ is not unsubstituted phenyl;

in case R¹ is hydroxy and R² is methyl and R³, R¹¹, R¹² and R¹³ represent hydrogen, R⁴ is not 4-chloro-phenyl; 2-naphthyl; 2-bromo-phenyl; 3-bromo-phenyl; 4-bromo-phenyl;

in case R¹ is hydroxy and R² is fluoro and R¹¹, R¹² and R¹³ represent hydrogen and R³ is methyl or ethyl, R⁴ is not 4-fluoro methyl;

in case R¹ and R¹² represent hydroxy and R¹¹ is chloro and R³ and R¹³ represent hydrogen and R² is n-butyl or (3-methyl)-butyl or n-pentyl, R⁴ is not 4-amino-2-hydroxy-phenyl;

in case R¹ and R¹² represent hydroxy and R² is ethyl or n-butyl or n-hexyl or (3-methyl)-butyl and R³, R¹¹ and R¹³ represent hydrogen, R⁴ is not unsubstituted phenyl, 4-amino-phenyl, 4-hydroxy-phenyl, 2-hydroxy-phenyl, 4-amino-2-hydroxy-phenyl,

and pharmaceutically acceptable salts thereof.

Preferred compounds are compounds of the formulae 2a-2e,

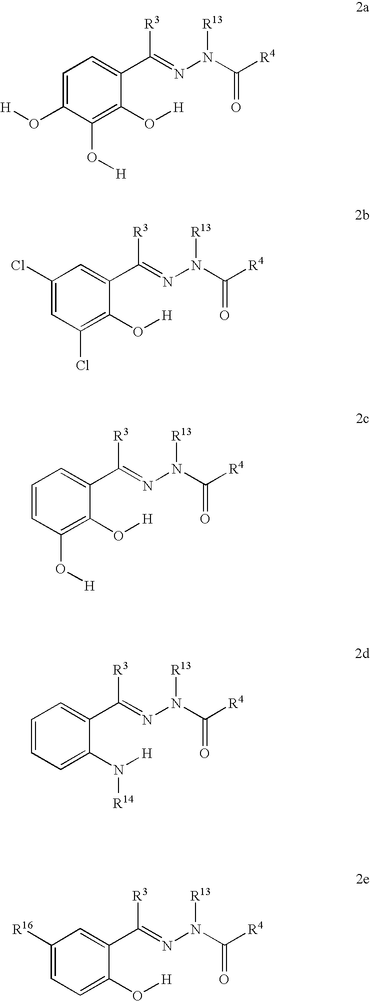

wherein R³, R¹³ and R⁴ have the meaning given in formula 1 and R¹⁴ is hydrogen, lower alkyl , formyl or acetyl and R¹⁶ is hydrogen, methyl, fluoro, chloro, hydroxy or ethyl and pharmaceutically acceptable salts thereof.

Very preferred compounds are compounds of the formulae 3a-3e,

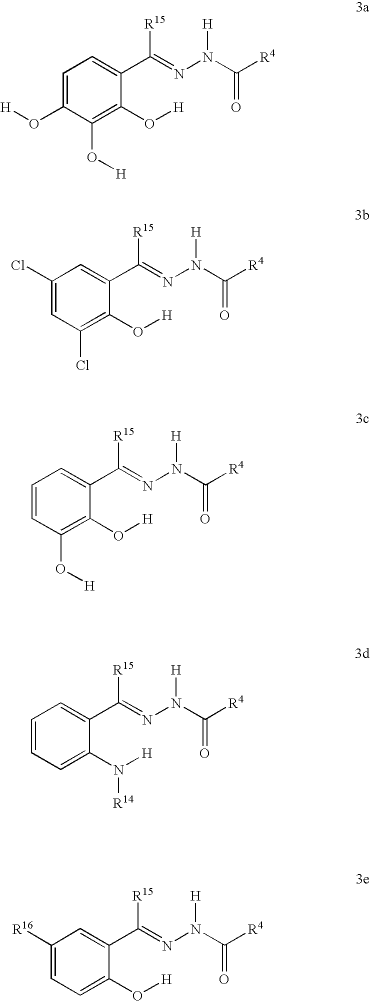

wherein R⁴ has the meaning given in formula 1 and R¹⁴ is hydrogen, lower alkyl, formyl or acetyl and R¹⁶ is hydrogen, methyl, fluoro, chloro, hydroxy or ethyl and R¹⁵ is hydrogen, methyl or ethyl and pharmaceutically acceptable salts thereof.

Especially preferred compounds are compounds of the formulae 4a-4f.

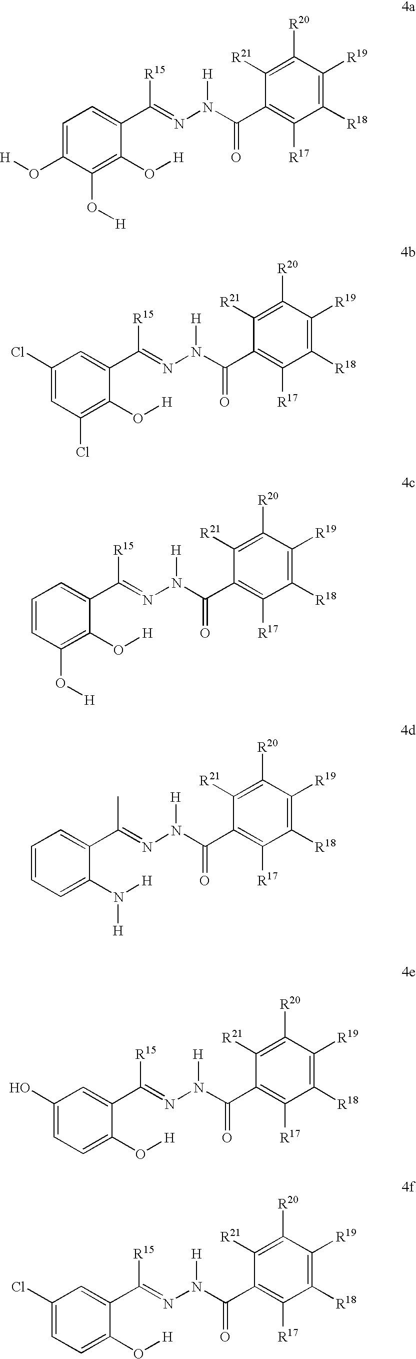

In formula 4a R 15 represents hydrogen, methyl or ethyl and, R¹⁷, R¹⁸, R¹⁹, R²⁰ and, R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R⁵ is methyl either one or two of the substituents R¹⁷, R¹⁸, R¹⁹, R²⁰, R²¹ represent N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy.

In formula 4b R¹⁵ represents hydrogen, methyl or ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R¹⁷ is N-pyrrolyl either one or two of the substituents R¹⁸, R¹⁹, R²⁰, R²¹ represent lower alkyl, hydroxy, lower alkoxy, fluoro, chloro, bromo, trifluoromethyl, amino, lower alkylamino, lower alkylendioxy.

In formula 4c R¹⁵ represents hydrogen, methyl or ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R¹⁵ is hydrogen and R¹⁷ is chloro either one or two of the substituents R¹⁸, R¹⁹, R²⁰, R²¹ represents, N-pyrrolyl, 2-pyrrolyl, 3-pyrrolyl, lower alkyl, hydroxy, lower alkoxy, fluoro, chloro, bromo, trifluoromethyl, amino, lower alkylamino or lower alkylendioxy.

In formula 4d R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R¹⁷ is hydrogen or hydroxy, either one or two of the substituents R¹⁸, R¹⁹, R²⁰, R²¹ represent N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy.

In formula 4e R¹⁵ represents hydrogen, methyl, ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy.

In formula 4f R¹⁵ represents hydrogen, methyl, ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R¹⁵ is hydrogen then at least one of the substituents R¹⁷, R¹⁸, R¹⁹, R²⁰ or R²¹ represents pyrrolyl, trifluoromethyl, or lower alkylamino

and pharmaceutically acceptable salts thereof.

Most preferred compounds are all end products mentioned in examples 1 to 53 including compounds of the formula 5a-e and pharmaceutically acceptable salts thereof.

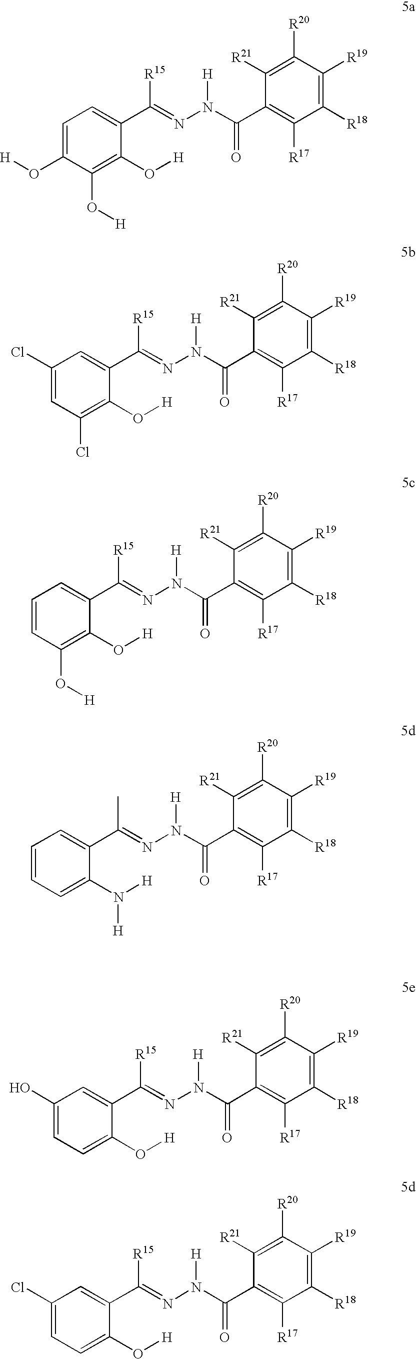

In formula 5a R¹⁵ represents hydrogen, methyl or ethyl and R¹⁷, R¹⁸, R¹⁹, R²⁰ and R²¹which may be the same or different, represent hydrogen, lower alkyl, hydroxy, lower alkoxy, fluoro, chloro, bromo, trifluoromethyl, lower alkylamino, lower alkylendioxy, with the proviso that one or two of the substituents R¹⁷, R¹⁸, R¹⁹, R²⁰ and R²¹ represent trifluoromethyl or chloro.

In formula 5b R¹⁵ represents hydrogen, methyl or ethyl and R¹⁷, R¹⁸, R¹⁹, R²⁰ and R²¹, which may be the same or different, represent hydrogen, lower alkyl, hydroxy, lower alkoxy, fluoro, chloro, bromo, trifluoromethyl, lower alkylamino, lower alkylendioxy, N-pyrrolyl, 2-pyrrolyl or 3-pyrrolyl, with the proviso that one or two of the substituents R¹⁷, R¹⁸, R¹⁹, R²⁰ and R²¹ represent N-pyrrolyl, 2-pyrrolyl or 3-pyrrolyl, in case R¹⁷ represents N-pyrrolyl, at least one of the substituents R¹⁸, R¹⁹, R²⁰ of R²¹ represents lower alkyl, hydroxy, lower alkoxy, fluoro, chloro, bromo, trifluoromethyl, lower alkylamino, lower alkylendioxy.

In formula 5c R¹⁵ represents hydrogen, methyl or ethyl and R¹⁷, R¹⁸, R¹⁹, R²⁰ and R²¹, which may be the same or different, represent hydrogen, lower alkyl, hydroxy, lower alkoxy, fluoro, chloro, bromo, trifluoromethyl, lower alkylamino, lower alkylendioxy, with the proviso that one or two of the substituents R¹⁷, R¹⁸, R¹⁹, R²⁰ and R²¹ represent chloro or trifluoromethyl. In formula 5d R¹⁷, R¹⁸, R¹⁹, R²⁰ and R²¹, which may be the same or different, represent hydrogen, lower alkyl, lower alkoxy, fluoro, chloro, bromo, trifluoromethyl, amino, lower alkylamino, lower alkylendioxy, with the proviso that one or two of the substituents R¹⁷, R¹⁸, R¹⁹, R²⁰ and R²¹ represent chloro, methoxy, methyl or trifluoromethyl.

In formula 5e R¹⁵ represents hydrogen, methyl, ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, with the proviso that one or two of the substituents R¹⁷, R¹⁸, R¹⁹, R²⁰ and R²¹ represent chloro, methoxy, methyl of trifluoromethyl.

In formula 5f R¹⁵ represents hydrogen, methyl, ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, with the proviso that in case R¹⁵ is hydrogen at least one of the substituents R¹⁷, R¹⁸, R¹⁹, R²⁰ and R²¹ represents N-pyrroly, 2-pyrrolyl, 3-pyrrolyl, trifluoromethyl or lower alkylamino.

In the definitions of the general formula 1—if not otherwise stated—the expression lower means straight and branched chain groups with one to seven carbon atoms, preferably 1 to 4 carbon atoms. Examples of lower alkyl and lower alkoxy groups are methyl, ethyl, n-propyl, isopropyl, n-butyl, isobutyl, sec.-butyl, tert.-butyl, pentyl, hexyl, heptyl, methoxy, ethoxy, propoxy, butoxy, iso-butoxy, sec.-butoxy and tert.-butoxy. The expression aryl represents unsubstituted as well as mono-, di- or tri-substituted aromatic rings with 6 to 10 carbon atoms like phenyl or naphthyl rings which may be substituted with halogen, hydroxy, lower alkyl, lower alkoxy, or lower alkylendioxy forming with the phenyl ring a five- or six-membered ring, trifluoromethyl, lower alkylamino.

The expression pharmaceutically acceptable salts encompasses either salts with inorganic acids or organic acids like hydrohalogenic acids, e.g. hydrochloric or hydrobromic acid; sulfuric acid, phosphoric acid, nitric acid, citric acid, formic acid, acetic acid, maleic acid, tartaric acid, methane sulfonic acid, p-toluene sulfonic acid and the like or in case the compound of formula 1 is acidic in nature with an inorganic base like an alkali or earth alkali base, e.g. sodium hydroxide, potassium hydroxide, calcium hydroxide, magnesium hydroxide etc.

Because of their ability to inhibit Gram positive and Gram negative bacteria, the described compounds can be used for the treatment of diseases which are associated with an infection by such type of pathogens. They are valuable anti-infectives.

The compounds can be administered orally, rectally, parenterally, e.g. by intravenous, intramuscular, subcutaneous, intrathecal or transdermal administration or sublingually or as ophthalmic preparation or administered as aerosol. Examples of applications are capsules, tablets, orally administered suspensions or solutions, suppositories, injections, eye-drops, ointments or aerosols/nebulizers.

Preferred applications are intravenous, intra-muscular, or oral administrations as well as eye drops. The dosage used depends upon the type of the specific active ingredient, the age and the requirements of the patient and the kind of application. Generally, dosages of 0.1-50 mg/kg body weight per day are considered. The preparations with compounds of formula 1 can contain inert or as well pharmacodynamically active excipients like sulphonamides. Tablets or granules, for Example, could contain a number of binding agents, filling excipients, carrier substances or diluents.

These compositions may be administered in enteral or oral form e.g. as tablets, dragees, gelatine capsules, emulsions, solutions or suspensions, in nasal form like sprays or rectally in form of suppositories. These compounds may also be administered in intramuscular, parenteral or intraveneous form, e.g. in form of injectable solutions.

These pharmaceutical compositions may contain the compounds of formula 1 as well as their pharmaceutically acceptable salts in combination with inorganic and/or organic excipients which are usual in the pharmaceutical industry like lactose, maize or derivatives thereof, talcum, stearinic acid or salts of these materials.

For gelatine capsules vegetable oils, waxes, fats, liquid or half-liquid polyols etc. may be used. For the preparation of solutions and syrups e.g. water, polyols, saccharose, glucose etc. are used. Injectables are prepared by using e.g. water, polyols, alcohols, glycerin, vegetable oils, lecithin, liposomes etc. Suppositories are prepared by using natural or hydrogenated oils, waxes, fatty acids (fats ), liquid or half-liquid polyols etc.

The compositions may contain in addition preservatives, stabilisation improving substances, viscosity improving or regulating substances, solubility improving substances, sweeteners, dyes, taste improving compounds, salts to change the osmotic pressure, buffer, anti oxidants etc.

The compounds of formula 1 may also be used in co-therapy with one or more other therapeutically used classes of antimicrobial substances, for example, beta-lactams e.g. penicillins and cephalosporins; glycopeptides; quinolones; tetracyclines; aminoglycosides; macrolides etc.

The dosage may vary within wide limits but should be adapted to the specific situation. In general the dosage given in oral form should daily be between about 3 mg and about 4 g, preferably between about 0.2 g and about 4 g, especially preferred between 0.2 g and 2 g per adult with a body weight of about 70 kg. The dosage should be administered preferably in 1 to 3 doses per day which are of equal weight. As usual children should receive lower doses which are adapted to body weight and age.

The invention also relates to a process for the manufacture of compounds of formula 1, which process comprises reacting

a) equimolar amounts of an aromatic carboxylic acid hydrazide and an aromatic aldehyde at ambient temperature, until the respective hydrazone precipitates, (Method A), or

b) equimolar amounts of an aromatic carboxylic acid hydrazide and an aromatic aldehyde at reflux temperature of the solvent, until the respective hydrazone precipitates (Method B).

A preferred solvent in step B is ethanol.

### EXAMPLES

The following examples illustrate the invention but do not limit the scope thereof. All temperatures are stated in degree centigrades.

### EXAMPLES

### Example 1 (Method A)

Benzoic acid hydrazide (1 mmol) and 2,5-dihydroxy-benzaldehyde (1 mmol) were suspended in 15 ml of ethanol. The mixture was stirred until N′-(2,5-dihydroxy-benzylidene)-benzohydrazide precipitated, which was filtered off and dried under vacuum.

### Example 2 (Method A)

2-(1H-indol-3-yl)-acetohydrazide (1 mmol) and 2-hydroxy-benzaldehyde (1 mmol) were suspended in 15 ml of ethanol. The mixture was stirred until N′-(2-hydroxy-benzylidene)-2-(1H-indol-3-yl)-acetohydrazide precipitated, which was filtered off and dried under vacuum.

### Example 3 (Method A)

1-Naphthoic acid hydrazide (1 mmol) and 2,5-dihydroxy-benzaldehyde (1 mmol) were suspended in 15 ml of ethanol. The mixture was stirred until N′-(2,5-dihydroxy-benzylidene)-naphthalene-1-carbohydrazide precipitated, which was filtered off and dried under vacuum.

### Example 4 (Method A)

3,4,5-Trimethoxy benzoic acid hydrazide (1 mmol) and 2,3,4-trihydroxy-benzaldehyde (1 mmol) were suspended in 15 ml of ethanol. The mixture was stirred until 3,4,5-trimethoxy-N′-(2,3,4-trihydroxy-benzylidene)-benzohydrazide precipitated, which was filtered off and dried under vacuum.

### Example 5 (Method A)

2-Amino-5-chloro benzoic acid hydrazide (1 mmol) and 2-hydroxy-benzaldehyde (1 mmol) were suspended in 15 ml of ethanol. The mixture was stirred until 2-amino-5-chloro-N′-(2-hydroxy-benzylidene)-benzohydrazide precipitated, which was filtered off and dried under vacuum.

### Example 6 (Method A)

3-Trifluoromethyl benzoic acid hydrazide (1 mmol) and 2,4-dihydroxy benzaldehyde (1 mmol) were suspended in 15 ml of ethanol. The mixture was stirred until 3-trifluoromethyl-N′-(2,4-dihydroxy-benzylidene)-benzohydrazide precipitated, which was filtered off and dried under vacuum.

### Example 7 (Method A)

3-Methoxy benzoic acid hydrazide (1 mmol) and 2-hydroxyacetophenone (1 mmol) were suspended in 15 ml of ethanol. The mixture was stirred until 3-methoxy-N′-[1-(2-hydroxy-phenyl)-ethylidene]-benzohydrazide precipitated, which was filtered off and dried under vacuum.

### Example 8 (Method A)

3-Methoxy benzoic acid hydrazide (1 mmol) and 2,5-dihydroxy benzaldehyde (1 mmol) were suspended in 15 ml of ethanol. The mixture was stirred until 3-methoxy-N′-(2,5-dihydroxy-benzylidene)-benzohydrazide precipitated, which was filtered off and dried under vacuum.

### Example 9 (Method A)

3,4-Dichloro benzoic acid hydrazide (1 mmol) and 2,3,4-trihydroxy benzaldehyde (1 mmol) were suspended in 15 ml of ethanol. The mixture was stirred until 3,4-dichloro-N′-(2,3,4-trihydroxy-benzylidene)-benzohydrazide precipitated, which was filtered off and dried under vacuum.

### Example 10 (Method A)

4-Chloro benzoic acid hydrazide (1 mmol) and 2,5-dihydroxy benzaldehyde (1 mmol) were suspended in 15 ml of ethanol. The mixture was stirred until 4-chloro-N′-(2,5-dihydroxy-benzylidene)-benzohydrazide precipitated, which was filtered off and dried under vacuum.

### Example 11 (Method A)

4-Hydroxy benzoic acid hydrazide (1 mmol) and 2,5-dihydroxy benzaldehyde (1 mmol) were suspended in 15 ml of ethanol. The mixture was stirred until 4-hydroxy-N′-(2,5-dihydroxy-benzylidene)-benzohydrazide precipitated, which was filtered off and dried under vacuum.

### Example 12 (Method A)

3,4-Dichloro benzoic acid hydrazide (1 mmol) and 2,5-dihydroxy benzaldehyde (1 mmol) were suspended in 15 ml of ethanol. The mixture was stirred until 3,4-dichloro-N′-(2,5-dihydroxy-benzylidene)-benzohydrazide precipitated, which was filtered off and dried under vacuum.

### Example 13 (Method A)

3-Chloro benzoic acid hydrazide (1 mmol) and 2,5-dihydroxy benzaldehyde (1 mmol) were suspended in 15 ml of ethanol. The mixture was stirred until 3-chloro-N′-(2,5-dihydroxy-benzylidene)-benzohydrazide precipitated, which was filtered off and dried under vacuum.

### Example 14 (Method A)

4-Hydroxy-3-methoxy benzoic acid hydrazide (1 mmol) and 5-chloro-2-hydroxy benzaldehyde (1 mmol) were suspended in 15 ml of ethanol. The mixture was stirred until 4-hydroxy-3-methoxy-N′-(5-chloro-2-hydroxy-benzylidene)-benzo-hydrazide precipitated, which was filtered off and dried under vacuum.

### Example 15 (Method A)

Benzoic acid hydrazide (1 mmol) and 2,5-dihydroxy acetophenone (1 mmol) were suspended in 15 ml of ethanol. The mixture was stirred until N′-[1-(2,5-dihydroxy-phenyl)-ethylidene]-benzohydrazide precipitated, which was filtered off and dried under vacuum.

### Example 16 (Method A)

4-Hydroxy-3-methoxy benzoic acid hydrazide (1 mmol) and 2,5-dihydroxy benzaldehyde (1 mmol) were suspended in 15 ml of ethanol. The mixture was stirred until N′-(2,5-dihydroxy-benzylidene)-4-hydroxy-3-methoxy-benzohydrazide precipitated, which was filtered off and dried under vacuum.

### Example 17 (Method A)

Benzoic acid hydrazide (1 mmol) and 2-hydroxy-5-methyl benzaldehyde (1 mmol) were suspended in 15 ml of ethanol. The mixture was stirred until N′-(2-hydroxy-5-methyl-benzylidene)-benzohydrazide precipitated, which was filtered off and dried under vacuum.

### Example 18 (Method A)

2-Methylamino-benzoic acid hydrazide (1 mmol) and 5-chloro-2-hydroxy benzaldehyde (1 mmol) were suspended in 15 ml of ethanol. The mixture was stirred until 2-methylamino-N′-(5-chloro-2-hydroxy-benzylidene)-benzohydrazide precipitated, which was filtered off and dried under vacuum.

### Example 19 (Method A)

2-Methylamino-benzoic acid hydrazide (1 mmol) and 2,5-dihydroxy benzaldehyde (1 mmol) were suspended in 15 ml of ethanol. The mixture was stirred until 2-methylamino-N′-(2,5-dihydroxy-benzylidene)-benzohydrazide precipitated, which was filtered off and dried under vacuum.

### Example 20 (Method A)

3-Methyl-benzoic acid hydrazide (1 mmol) and 5-chloro-2-hydroxy benzaldehyde (1 mmol) were suspended in 15 ml of ethanol. The mixture was stirred until 3-methyl-N′-(5-chloro-2-hydroxy-benzylidene)-benzohydrazide precipitated, which was filtered off and dried under vacuum.

### Example 21 (Method A)

3-Trifluoromethyl-benzoic acid hydrazide (1 mmol) and 5-chloro-2-hydroxy benzaldehyde (1 mmol) were suspended in 15 ml of ethanol. The mixture was stirred until 3-trifluoromethyl-N′-(5-chloro-2-hydroxy-benzylidene)-benzohydrazide precipitated, which was filtered off and dried under vacuum.

### Example 22 (Method A)

2-Methylamino-benzoic acid hydrazide (1 mmol) and 2-hydroxy acetophenone (1 mmol) were suspended in 15 ml of ethanol. The mixture was stirred until 2-methylamino-N′-[1-(2-hydroxy-phenyl)-ethylidene]-benzohydrazide precipitated, which was filtered off and dried under vacuum.

### Example 23 (Method A)

Benzoic acid hydrazide (1 mmol) and 2-acetamino acetophenone (1 mmol) were suspended in 15 ml of ethanol. The mixture was stirred until N-[2-[1-(2-benzoyl-hydrazono)-ethyl]-phenyl]-acetamide precipitated, which was filtered off and dried under vacuum.

### Example 24 (Method B)

4-Chlorobenzhydrazide (1 mmol) and 2-amino acetophenone (1 mmol) were dissolved in 20 ml of ethanol. The mixture was refluxed for 60 hours and stirring was then continued at ambient temperature. After several days 4-chloro-N′-[1-(2-amino-phenyl)-ethylidene]-benzohydrazide precipitated. The product was filtered and dried under vacuum.

### Example 25 (Method B)

3-Methoxy benzhydrazide (1 mmol) and 2-amino acetophenone (1 mmol) were dissolved in 20 ml of ethanol. The mixture was refluxed for 60 hours and stirring was then continued at ambient temperature. After several days 3-methoxy-N′-[1-(2-amino-phenyl)-ethylidene]-benzohydrazide precipitated. The product was filtered and dried under vacuum.

### Example 26 (Method A)

Benzoic acid hydrazide (1 mmol) and 2,3-dihydroxy benzaldehyde (1 mmol) were suspended in 15 ml of ethanol. The mixture was stirred until N′-(2,3-dihydroxy-benzylidene)-benzohydrazide precipitated, which was filtered off and dried under vacuum.

### Example 27 (Method A)

3-Methoxy benzoic acid hydrazide (1 mmol) and 2-hydroxy benzaldehyde (1 mmol) were suspended in 15 ml of ethanol. The mixture was stirred until 3-methoxy-N′-(2-hydroxy-benzylidene)-benzohydrazide precipitated, which was filtered off and dried under vacuum.

### Example 28 (Method A)

Benzoic acid hydrazide (1 mmol) and 2,3,4-trihydroxy benzaldehyde (1 mmol) were suspended in 15 ml of ethanol. The mixture was stirred until N′-(2,3,4-trihydroxy-benzylidene)-benzohydrazide precipitated, which was filtered off and dried under vacuum.

### Example 29 (Method A)

Benzoic acid hydrazide (1 mmol) and 2,3,5-trihydroxy benzaldehyde (1 mmol) were suspended in 15 ml of ethanol. The mixture was stirred until N′-(2,3,5-trihydroxy-benzylidene)-benzohydrazide precipitated, which was filtered off and dried under vacuum.

### Example 30 (Method A)

3,4,5-Trimethoxy benzoic acid hydrazide (1 mmol) and 2,3,5-trihydroxy benzaldehyde (1 mmol) were suspended in 15 ml of ethanol. The mixture was stirred until 3,4,5-trimethoxy-N′-(2,4,5-trihydroxy-benzylidene)-benzohydrazide precipitated, which was filtered off and dried under vacuum.

### Example 31 (Method A)

4-Bromo benzoic acid hydrazide (1 mmol) and 2-hydroxy benzaldehyde (1 mmol) were suspended in 15 ml of ethanol. The mixture was stirred until 4-bromo-N′-(2-hydroxy-benzylidene)-benzohydrazide precipitated, which was filtered off and dried under vacuum.

### Example 32 (Method A)

3-Trifluoromethyl benzoic acid hydrazide (1 mmol) and 2-hydroxy benzaldehyde (1 mmol) were suspended in 15 ml of ethanol. The mixture was stirred until 3-trifluoromethyl-N′-(2-hydroxy-benzylidene)-benzohydrazide precipitated, which was filtered off and dried under vacuum.

### Example 33 (Method A)

3-Methyl benzoic acid hydrazide (1 mmol) and 2,5-dihydroxy benzaldehyde (1 mmol) were suspended in 15 ml of ethanol. The mixture was stirred until 3-methyl-N′-(2,5-dihydroxy-benzylidene)-benzohydrazide precipitated, which was filtered off and dried under vacuum.

### Example 34 (Method A)

3-Trifluoromethyl benzoic acid hydrazide (1 mmol) and 2,5-dihydroxy benzaldehyde (1 mmol) were suspended in 15 ml of ethanol. The mixture was stirred until 3-trifluoromethyl-N′-(2,5-dihydroxy-benzylidene)-benzohydrazide precipitated, which was filtered off and dried under vacuum.

### Example 35 (Method B)

4-Hydroxy benzhydrazide (1 mmol) and 2,5-dihydroxy acetophenone (1 mmol) were dissolved in 20 ml of ethanol. The mixture was refluxed for 60 hours and stirring was then continued at ambient temperature. After several days 4-hydroxy-N′-[1-(2,5-dihydroxy-phenyl)-ethylidene]-benzohydrazide precipitated. The product was filtered and dried under vacuum.

### Example 36 (Method A)

4-Chloro benzoic acid hydrazide (1 mmol) and 2-hydroxy-3-chloro benzaldehyde (1 mmol) were suspended in 15 ml of ethanol. The mixture was stirred until 4-chloro-N′-(2-hydroxy-3-chloro-benzylidene)-benzohydrazide precipitated, which was filtered off and dried under vacuum.

### Example 37 (Method A)

4-Chloro benzoic acid hydrazide (1 mmol) and 2,4-dihydroxy benzaldehyde (1 mmol) were suspended in 15 ml of ethanol. The mixture was stirred until 3-chloro-N′-(2,4-dihydroxy-benzylidene)-benzohydrazide precipitated, which was filtered off and dried under vacuum.

### Example 38 (Method A)

3-Chloro benzoic acid hydrazide (1 mmol) and 2-hydroxy-5-chloro benzaldehyde (1 mmol) were suspended in 15 ml of ethanol. The mixture was stirred until 4-chloro-N′-(2-hydroxy-5-chloro-benzylidene)-benzohydrazide precipitated, which was filtered off and dried under vacuum.

### Example 39 (Method A)

4-Methoxy benzoic acid hydrazide (1 mmol) and 2,3,4-trihydroxy benzaldehyde (1 mmol) were suspended in 15 ml of ethanol. The mixture was stirred until 4-methoxy-N′-(2,3,4-trihydroxy-benzylidene)-benzohydrazide precipitated, which was filtered off and dried under vacuum.

### Example 40 (Method A)

3,4-Dichloro benzoic acid hydrazide (1 mmol) and 2,3-dihydroxy benzaldehyde (1 mmol) were suspended in 15 ml of ethanol. The mixture was stirred until 3,4-dichloro-N′-(2,3-dihydroxy-benzylidene)-benzohydrazide precipitated, which was filtered off and dried under vacuum.

### Example 41 (Method A)

3,5-Bis-(trifluoromethyl)-benzoic acid hydrazide (1 mmol) and 2,3,4-trihydroxy benzaldehyde (1 mmol) were suspended in 15 ml of ethanol. The mixture was stirred until 3,5-Bis-(trifluoromethyl)-N′-(2,3,4-trihydroxy-benzylidene)-benzo-hydrazide precipitated, which was filtered off and dried under vacuum.

### Example 42 (Method A)

3-Chloro-2-pyrrol-1-yl benzoic acid hydrazide (1 mmol), of which the synthesis is described in examples 54-56, and 2,3,4-trihydroxy benzaldehyde (1 mmol) were suspended in 15 ml of ethanol. The mixture was stirred until 3-chloro-2-pyrrol-1-yl-N′-(2,3,4-trihydroxy-benzylidene)-benzohydrazide precipitated, which was filtered off and dried under vacuum.

### Example 43 (Method A)

3-Chloro-2-pyrrol-1-yl benzoic acid hydrazide (1 mmol), of which the synthesis is described in examples 54-56, and 2-hydroxy-3,5-dichloro benzaldehyde (1 mmol) were suspended in 15 ml of ethanol. The mixture was stirred until 3-chloro-2-pyrrol-1-yl-N′-(2-hydroxy-3,5-dichloro-benzylidene)-benzohydrazide precipitated, which was filtered off and dried under vacuum.

### Example 44 (Method A)

2-Pyrrol-1-yl benzoic acid hydrazide (1 mmol) and 2,3,5-trihydroxy benzaldehyde (1 mmol) were suspended in 15 ml of ethanol. The mixture was stirred until 2-pyrrol-1-yl-N′-(2,3,5-trihydroxy-benzylidene)-benzohydrazide precipitated, which was filtered off and dried under vacuum.

### Example 45 (Method A)

4-Chloro-3-trifluoromethyl benzoic acid hydrazide (1 mmol) and 2,3,4-trihydroxy benzaldehyde (1 mmol) were suspended in 15 ml of ethanol. The mixture was stirred until 4-chloro-3-trifluoromethyl-N′-(2,3,4-dihydroxy-benzylidene)-benzo-hydrazide precipitated, which was filtered off and dried under vacuum.

### Example 46 (Method A)

4-Chloro-3-trifluoromethyl benzoic acid hydrazide (1 mmol) and 2-hydroxy-3,5-dichloro benzaldehyde (1 mmol) were suspended in 15 ml of ethanol. The mixture was stirred until 4-chloro-3-trifluoromethyl-N′-(2-hydroxy-3,5-dichloro-benzylidene)-benzohydrazide precipitated, which was filtered off and dried under vacuum.

### Example 47 (Method A)

4-Chloro benzoic acid hydrazide (1 mmol) and 2,4,5-trihydroxy benzaldehyde (1 mmol) were suspended in 15 ml of ethanol. The mixture was stirred until 4-chloro-—N′-(2,3,4-trihydroxy benzylidene)-benzohydrazide precipitated, which was filtered off and dried under vacuum.

### Example 48 (Method A)

Benzoic acid hydrazide (1 mmol) and 2-hydroxy-3,5-dichloro benzaldehyde (1 mmol) were suspended in 15 ml of ethanol. The mixture was stirred until N′-(2-hydroxy-3,5-dichloro-benzylidene)-benzohydrazide precipitated, which was filtered off and dried under vacuum.

### Example 49 (Method A)

3-Chloro benzoic acid hydrazide (1 mmol) and 2,3,4-trihydroxy benzaldehyde (1 mmol) were suspended in 15 ml of ethanol. The mixture was stirred until 3-chloro-N′-(2,3,4-trihydroxy benzylidene)-benzohydrazide precipitated, which was filtered off and dried under vacuum.

### Example 50 (Method A)

3-Trifluoromethyl benzoic acid hydrazide (1 mmol) and 2,3,5-trihydroxy benzaldehyde (1 mmol) were suspended in 15 ml of ethanol. The mixture was stirred until 3-trifluoromethyl-N′-(2,3,5-trihydroxybenzylidene)-benzohydrazide precipitated, which was filtered off and dried under vacuum.

### Example 51 (Method A)

3-Trifluoromethyl benzoic acid hydrazide (1 mmol) and 2,3,4-trihydroxy benzaldehyde (1 mmol) were suspended in 15 ml of ethanol. The mixture was stirred until 3-trifluoromethyl-N′-(2,3,4-trihydroxybenzylidene)-benzohydrazide precipitated, which was filtered off and dried under vacuum.

### Example 52 (Method A)

3,4-Dichloro benzoic acid hydrazide (1 mmol) and 2,3,4-trihydroxy acetophenone (1 mmol) were suspended in 15 ml of ethanol. The mixture was stirred until 3,4-dichloro-N′-[1-(2,3,4-dihydroxy-phenyl)-ethylidene]-benzohydrazide precipitated, which was filtered off and dried under vacuum.

### Example 53 (Method A)

3,4-Dichloro benzoic acid N-methyl hydrazide (1 mmol), of which the synthesis is described in example 57, and 2,3,4-trihydroxy benzaldehyde (1 mmol) were suspended in 15 ml of ethanol. The mixture was stirred until 3,4-dichloro-N-methyl-N′-(2,3,4-trihydroxy-benzylidene)-benzohydrazide precipitated, which was filtered off and dried under vacuum.

### Example 54

Synthesis of 3-chloro-2-pyrrol-1-yl-benzoic acid

3-Chloro-2-amino benzoic acid (2 g) and 2,5-dimethyl-tetrahydrofuran (1.6 g) were dissolved in dioxane (10 ml). To this mixture pyridine hydrochloride (700 mg) was added. The mixture was stirred at room temperture under an argon atmosphere for 16 hours followed by 3 hours at 80° C. The solvents were completely removed in vacuo and the residue was separated between ethyl ether and water. The organic phase was washed with brine, dried with magnesium sulfate. The solvents were completely removed in vacuo. 3-Chloro-2-pyrrol-1-yl-benzoic acid was obtained by crystalization in ethyl acetate/hexane. After the crystals were dissolved in ethyl acetate and this solution was filtered over active carbon, pure 3-chloro-2-pyrrol-1-yl-benzoic acid was obtained by removal of the solvent.

MS: ESI-220u, 222u

### Example 55

Synthesis of 3-chloro-2-pyrrol-1-yl-benzoic acid methyl ester

3-Chloro-2-pyrrol-1-yl-benzoic acid (1.6 g) was dissolved in methanol (30 ml) and concentrated sulfuric acid (0.5 ml) was added. The mixture was kept under reflux for 5.5 hours, cooled to room temperature, cautiously poured on aqueous sodium hydrogencarbonate solution. To this mixture ethyl acetate was added, the layers were separated, the organic layer was washed with brine, dried with magnesium sulfate and the solvents were removed in vacuo. The compound was pure on TLC.

TLC: (plates: Machery Nagel polygram SIL/UV, solvent hexane/ethyl acetate 4/1)

Rf 0.5

IR: film C═O 1728.7/cm

### Example 56

Synthesis of 3-chloro-2-pyrrol-1-yl-benzoic acid hydrazide

3-Chloro-2-pyrrol-1-yl-benzoic acid methyl ester (1.45 g) and hydrazine hydrate (80% in water, 750 mg) were dissolved in ethanol (10 ml) and refluxed over night. The solvents were removed to obtain a pure solid.

MS ESI+236u, 238 u

### Example 57

Synthesis of 3,4-dichloro-benzoic acid N-methyl hydrazide

3,4-Dichloro-benzoyl chloride (4.18 g) was dissolved in methylene chloride (20 ml). To this solution methyl hydrazine (4.0 ml) was added. After stirring the solution for 90 minutes the mixture was distributed between methylene chloride and water. The layers were separated, the aqueous layer was extracted several times with methylene chloride, the organic layers were combined, and the solvents were removed in vacuo. After column chromatography pure compound was obtained.

TLC: (plates: Machery Nagel polygram SIL/UV, solvent hexane/ethyl acetate 3/1)

Rf 0.15

The identity and purity of the end products of examples 1-53 was examined by MS-spectroscopy. The applied method was APCI, if not otherwise stated as ESI.

m/e values for the positive and negative ion signals which are set forth in the table 3 below.

| | | | molecular | MS pos | MS neg |
|---|---|---|---|---|---|
| | | | weight in | mode | mode |
| Compounds | Example | Method | g/mol | m/e in u | m/e in u |
| N′-(2,5-Dihydroxy-benzylidene)- | 1 | A | 256 | 257 | 255 |
| benzohydrazide | | | | | |
| N′-(2-Hydroxy-benzylidene)-2- | 2 | A | 293 | 294 | 292 |
| (1H-indol-3-yl)-acetohydrazide | | | | | |
| N′-(2,5-Dihydroxy-benzylidene)- | 3 | A | 306 | 307 | 305 |
| naphthalene-1-carbohydrazide | | | | | |
| 3,4,5-Trimethoxy-N′-(2,3,4- | 4 | A | 362 | 363 | 361 |
| trihydroxy-benzylidene)- | | | | | |
| benzohydrazide | | | | | |
| 2-Amino-5-chloro-N′-(2- | 5 | A | 289.7 | 290 | 288 |
| hydroxy-benzylidene)- | | | | | |
| benzohydrazide | | | | | |
| 3-Trifluoromethyl-N′-(2,4- | 6 | A | 324 | 325 (ESI) | nd |
| dihydroxy-benzylidene)- | | | | | |
| benzohydrazide | | | | | |
| 3-Methoxy-N′-[1-(2-hydroxy- | 7 | B | 284 | 285 | 283 |
| phenyl)-ethylidene]- | | | | | |
| benzohydrazide | | | | | |
| 3-Methoxy-N′-(2,5-dihydroxy- | 8 | A | 286 | 287 | 285 |
| benzylidene)-benzohydrazide | | | | | |
| 3,4-Dichloro-N′-(2,3,4- | 9 | A | 341 | 341, 343, | nd |
| trihydroxy-benzylidene)- | | | | 345 (ESI) | |
| benzohydrazide | | | | | |
| 4-Chloro-N′-(2,5-dihydroxy- | 10 | A | 290.7 | 291 | 289 |
| benzylidene)-benzohydrazide | | | | | |
| 4-Hydroxy-N′-(2,5-dihydroxy- | 11 | A | 272 | 273 | 271 |
| benzylidene)-benzohydrazide | | | | | |
| 3,4-Dichloro-N′-(2,5-dihydroxy- | 12 | A | 325 | 325/327 | 323/325 |
| benzylidene)-benzohydrazide | | | | | |
| 3-Chloro-N′-(2,5-dihydroxy- | 13 | A | 290 | 291 | 289 |
| benzylidene)-benzohydrazide | | | | | |
| 4-Hydroxy-3-methoxy-N′-(5- | 14 | A | 320.7 | 321 | 319 |
| chloro-2-hydroxy-benzylidene)- | | | | | |
| benzohydrazide | | | | | |
| N′-[1-(2,5-Dihydroxy-phenyl)- | 15 | A | 270 | 271 | 269 |
| ethylidene]-benzohydrazide | | | | | |
| N′-(2,5-Dihydroxy-benzylidene)- | 16 | A | 302 | 303 | 301 |
| 4-hydroxy-3-methoxy- | | | | | |
| benzohydrazide | | | | | |
| N′-(2-Hydroxy-5-methyl- | 17 | A | 254 | 255 | 253 |
| benzylidene)-benzohydrazide | | | | | |
| 2-Methylamino-N′-(5-Chloro-2- | 18 | A | 303.7 | 304 | 302 |
| hydroxy-benzylidene)- | | | | | |
| benzohydrazide | | | | | |
| 2-Methylamino-N′-(2,5- | 19 | A | 285 | 286 | 284 |
| dihydroxy-benzylidene)- | | | | | |
| benzohydrazide | | | | | |
| 3-Methyl-N′-(5-chloro-2- | 20 | A | 288.7 | 289 | 287 |
| hydroxy-benzylidene)- | | | | | |
| benzohydrazide | | | | | |
| 3-Trifluoromethyl-N′-(5-chloro- | 21 | A | 342.7 | 343 | 341 |
| 2-hydroxy-benzylidene)- | | | | | |
| benzohydrazide | | | | | |
| 2-Methylamino-N′-[1-(2- | 22 | A | 283 | 284 | 282 |
| hydroxy-phenyl)-ethylidene]- | | | | | |
| benzohydrazide | | | | | |
| N-[2-[1-(2-Benzoyl-hydrazono)- | 23 | A | 295 | 296 | 294 |
| ethyl]-phenyl]-acetamide | | | | | |
| 4-Chloro-N′-[1-(2-amino- | 24 | B | 287.7 | 288 | 286 |
| phenyl)-ethylidene]- | | | | | |
| benzohydrazide | | | | | |
| 3-Methoxy-N′-[1-(2-amino- | 25 | B | 283 | 284 | 282 |
| phenyl)-ethylidene]- | | | | | |
| benzohydrazide | | | | | |
| N′-(2,3-Dihydroxy-benzylidene)- | 26 | A | 256 | nd | 255 |
| benzohydrazide | | | | | |
| 3-Methoxy-N′-(2-hydroxy- | 27 | A | 270 | 271 | 269 |
| benzylidene)-benzohydrazide | | | | | |
| N′-(2,3,4-Trihydroxy- | 28 | A | 272 | 273 | 271 |
| benzylidene)-benzohydrazide | | | | | |
| N′-(2,4,5-Trihydroxy- | 29 | A | 272 | 273 | 271 |
| benzylidene)-benzohydrazide | | | | | |
| 3,4,5-Trimethoxy-N′-(2,4,5- | 30 | A | 362 | 363 | 361 |
| trihydroxy-benzylidene)- | | | | | |
| benzohydrazide | | | | | |
| 4-Bromo-N′-(2-hydroxy- | 31 | A | 319 | 319, 321 | 317, 319 |
| benzylidene)-benzohydrazide | | | | | |
| 3-Trifluoromethyl-N′-(2-hydroxy- | 32 | A | 308 | 309 | 307 |
| benzylidene)-benzohydrazide | | | | | |
| 3-Methyl-N′-(2,5-dihydroxy- | 33 | A | 270 | 271 | 269 |
| benzylidene)-benzohydrazide | | | | | |
| 3-Trifluoromethyl-N′-(2,5- | 34 | A | 324 | 325 | 323 |
| dihydroxy-benzylidene)- | | | | | |
| benzohydrazide | | | | | |
| 4-Hydroxy-N′-[1-(2,5-dihydroxy- | 35 | B | 286 | nd | 285 (ESI) |
| phenyl)-ethylidene]- | | | | | |
| benzohydrazide | | | | | |
| 4-chloro-N′-(2-hydroxy-3-chloro- | 36 | A | 274.7 | nd | 273, 275 |
| benzylidene)-benzohydrazide | | | | | |
| 4-Chloro-N′-(2,4-dihydroxy- | 37 | A | 289 | nd | 289, 291 |
| benzylidene)-benzohydrazide | | | | | |
| 3-Chloro-N′-(2-hydroxy-5- | 38 | A | 309 | nd | 307, 309 |
| chloro-benzylidene)- | | | | | |
| benzohydrazide | | | | | |
| 4-Methoxy-N′-(2,3,4-trihydroxy- | 39 | A | 302 | 303 (ESI) | nd |
| benzylidene)-benzohydrazide | | | | | |
| 3,4-Dichloro-N′-(2,3-dihydroxy- | 40 | A | 325 | 325, 357 | nd |
| benzylidene)-benzohydrazide | | | | (ESI) | |
| 3,5-Bis-(trifluoromethyl)-N′- | 41 | A | 308 | 309 (ESI) | nd |
| (2,3,4-trihydroxy-benzylidene)- | | | | | |
| benzohydrazide | | | | | |
| 3-Chloro-2-pyrrol-1-yl-N′-(2,3,4- | 42 | A | 371.7 | nd | 370, 372 |
| trihydroxy-benzylidene)- | | | | | (ESI) |
| benzohydrazide | | | | | |
| 3-Chloro-2-pyrrol-1-yl-N′-(2- | 43 | A | 408.7 | nd | 406, 408, |
| hydroxy-3,5-dichloro- | | | | | 410 (ESI) |
| benzylidene)-benzohydrazide | | | | | |
| 2-Pyrrol-1-yl-N′-(2,4,5- | 44 | A | 337 | nd | 336(ESI) |
| trihydroxy-benzylidene)- | | | | | |
| benzohydrazide | | | | | |
| 4-Chloro-3-trifluoromethyl-N′- | 45 | A | 374.7 | nd | 373, 375 |
| (2,3,4-trihydroxy-benzylidene)- | | | | | (ESI) |
| benzohydrazide | | | | | |
| 4-Chloro-3-trifluoromethyl-N′-(2- | 46 | A | 411.6 | nd | 409, 411, |
| hydroxy-3,5-dichloro- | | | | | 413, 414 |
| benzylidene)-benzohydrazide | | | | | (ESI) |
| 4-Chloro-N′-(2,4,5-trihydroxy- | 47 | A | 306.7 | 307, 309 | 305, 307 |
| benzylidene)-benzohydrazide | | | | | |
| N′-(2-Hydroxy-3,5-dichloro- | 48 | A | 309 | 309, 311, | 307, 309, |
| benzylidene)-benzohydrazide | | | | 313 | 311 |
| 3-Chloro-N′-(2,3,4-trihydroxy- | 49 | A | 306.7 | 307, 309 | nd |
| benzylidene)-benzohydrazide | | | | (ESI) | |
| 3-Trifluoromethyl-N′-(2,4,5- | 50 | A | 340 | 341 (ESI) | nd |
| trihydroxy-benzylidene)- | | | | | |
| benzohydrazide | | | | | |
| 3-Trifluoromethyl-N′-(2,3,4- | 51 | A | 340 | 341 (ESI) | nd |
| trihydroxy-benzylidene)- | | | | | |
| benzohydrazide | | | | | |
| 3,4-Dichloro-N′-[1-(2,3,4- | 52 | A | 355 | nd | 355, 357, |
| dihydroxy-phenyl)-ethylidene]- | | | | | 359 (ESI) |
| benzohydrazide | | | | | |
| 3,4-Dichloro-N-methyl-N′-(2,3,4- | 53 | A | 355 | nd | 353, 355, |
| trihydroxy-benzylidene)- | | | | | 357 |
| benzohydrazide | | | | | |

nd means not determined

List of abbreviations

| | |
|---|---|
| APCI | atmospheric pressure ionization |
| ESI | electro spray ionization |
| IR | infrared spectroscopy |
| MIC | minimal inhibitory concentration |
| MS | mass spectroscopy |
| TLC | thin layer chromatography |

1. Compounds of the general formula 1, 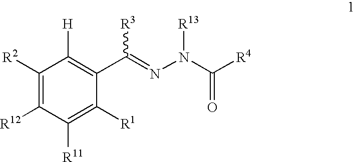
wherein R¹ represents lower alkyl-carbonylamino; formylamino; amino; hydroxy;
R² represents hydrogen; hydroxy; lower alkyl; fluoro; chloro;
R³ represents hydrogen; methyl; ethyl; isopropyl;
R¹¹ represents hydrogen; hydroxy; lower alkyl; lower alkoxy; fluoro; chloro; amino;
R¹² represents hydrogen; hydroxy; lower alkyl; lower alkoxy; fluoro; chloro; amino
R¹³ represents hydrogen; lower alkyl
R⁴ represents aryl; arylmethyl; indoyl methyl; mono-, di- or triN′- substituted aryl, arylmethyl, which substituents may lower alkyl, hydroxy, lower alkoxy, fluoro, chloro, bromo, trifluoromethyl, amino, lower alkylamino, lower alkylendioxy, N-pyrrolyl, 2-pyrrolyl, 3, pyrrolyl and which substituents may be the same or different;
in case R¹ represents amino and R², R¹¹, R¹², R¹³ and R³ represent hydrogen, R⁴ is not unsubstituted phenyl; phenylmethyl; 2-amino-phenyl; 2-hydroxy-phenyl; 4-chloro-phenyl;
in case R¹ represents amino and R², R¹¹, R¹² and R¹³ represent hydrogen and R³ represents methyl, R⁴ is not unsubstituted phenyl; 2-hydroxy-phenyl;
in case R¹ represents methyl-carbonylamino and R², R³, R¹¹, R¹³ and R¹² represent hydrogen, R⁴ is not 4-hydroxy-3-methoxy-phenyl;
in case R¹ is hydroxy and R², R¹¹, R¹² and R¹³ represent hydrogen and R³ represents methyl, R⁴ is not unsubstituted phenyl; 4-methyl-phenyl; 2-methyl-phenyl; 2-hydroxy-phenyl; 4-methoxy-phenyl; 4-chloro-phenyl; 2-chloro-phenyl; 2,4,6-trimethyl-phenyl;
in case R¹ is hydroxy and R², R¹¹, R¹² and R¹³ represent hydrogen and R³ represents ethyl, R⁴ is not unsubstitued phenyl or 2-hydroxy-phenyl;
in case R¹ is hydroxy and R², R¹¹, R¹² and R³ represent hydrogen and R¹³ represents methyl, R⁴ is not unsubstituted phenyl;
in case R¹ is hydroxy and R², R¹¹, R¹², R¹³ and R³ represent hydrogen, R⁴ is phenyl substituted with 2-trifluoromethyl, 3-trifluoromethyl, 3-methoxy or (2-amino-5-chloro);
in case R¹ and R¹¹ represent hydroxy and R², R³, R¹² and R¹³ represent hydrogen, R⁴ is not 2-chloro-phenyl;
in case R¹ is hydroxy and R¹¹ is methoxy and R², R³, R¹² and R¹³ represent hydrogen, R⁴ is not unsubstituted phenyl; 2-hydroxy-phenyl; 2-chloro-phenyl; 4-hydroxy-3-methoxy-phenyl; 5-chloro-2-hydroxy-phenyl; 2-(3-hydroxy)-naphthyl; 2,4-dichloro-phenyl; 4-amino-3,5-dichloro-phenyl; 5-bromo-2-hydroxy-phenyl;
in case R¹, R¹¹ and R¹² represent hydroxy and R² and R¹³ represent hydrogen and R³ is methyl, R⁴ is not unsubstituted phenyl;
in case R¹ and R¹² represent hydroxy and R², R³, R¹¹ and R¹³ represent hydrogen, R⁴ is not unsubstituted phenyl; 2-hydroxy-phenyl; 4-methoxy-phenyl; 4-hydroxy-3-methoxy-phenyl; 2,4-dichloro-phenyl;
in case R¹ and R¹² represent hydroxy and R², R¹¹ and R¹³ represent hydrogen and R³ is methyl, R⁴ is not unsubstituted phenyl; 2-hydroxy-phenyl;
in case R¹ is hydroxy and R¹² is methoxy and R², R³, R¹¹ and R¹³ represent hydrogen, R⁴ is not 4-hydroxy-3-methoxy-phenyl;
in case R¹ is hydroxy and R¹² is methoxy and R², R¹¹ and R¹³ represent hydrogen and R³ is methyl, R⁴ is not unsubstituted phenyl;
in case R¹ is hydroxy and R² is chloro and R³, R¹¹, R¹² and R¹³ represent hydrogen, R⁴ is not unsubstituted phenyl; 2-methyl-phenyl; 2-hydroxy-phenyl; 4-hydroxy-phenyl; 4-methoxy-phenyl; 4-chloro-phenyl; 5-chloro-2-hydroxy-phenyl; 2-hydroxy naphth-1-yl; 3-hydroxy naphth-2-yl; 2,4-dichloro-phenyl; 3,4-dichloro-phenyl; 3,4,5-trihydroxy-phenyl; 5-bromo-2-hydroxy-phenyl;
in case R¹ is hydroxy and R² and R¹¹ represent chloro and R³, R¹² and R¹³ represent hydrogen, R⁴ is not 2-hydroxy-phenyl; 5-chloro-2-hydroxy-phenyl; 3-hydroxy-naphth-2-yl; 2-hydroxy-3,5-dichloro-phenyl; 5-bromo-2-hydroxy-phenyl; 3,5-dibromo-2-hydroxy-phenyl; N-pyrrolyl;
in case R¹ is hydroxy and R² and R³ represent methyl and R¹¹, R¹² and R¹³ represent hydrogen, R⁴ is not unsubstituted phenyl;
in case R¹ is hydroxy and R² is methyl and R³, R¹¹, R¹² and R¹³ represent hydrogen, R⁴ is not 4-chloro-phenyl; 2-naphthyl; 2-bromo-phenyl; 3-bromo-phenyl; 4-bromo-phenyl;
in case R¹ is hydroxy and R² is fluoro and R¹¹, R¹² and R¹³ represent hydrogen and R³ is methyl or ethyl, R⁴ is not 4-fluoro methyl;
in case R¹ and R¹² represent hydroxy and R¹¹ is chloro and R³ and R¹³ represent hydrogen and R² is n-butyl or (3-methyl)-butyl or n-pentyl, R⁴ is not 4-amino-2-hydroxy-phenyl;
in case R¹ and R¹² represent hydroxy and R² is ethyl or n-butyl or n-hexyl or (3-methyl)-butyl and R³, R¹¹ and R¹³ represent hydrogen, R⁴ is not unsubstituted phenyl, 4-amino-phenyl, 4-hydroxy-phenyl, 2-hydroxy-phenyl, 4-amino-2-hydroxy-phenyl,
and pharmaceutically acceptable salts thereof.

2. Compounds of the formulae 2a-2e, 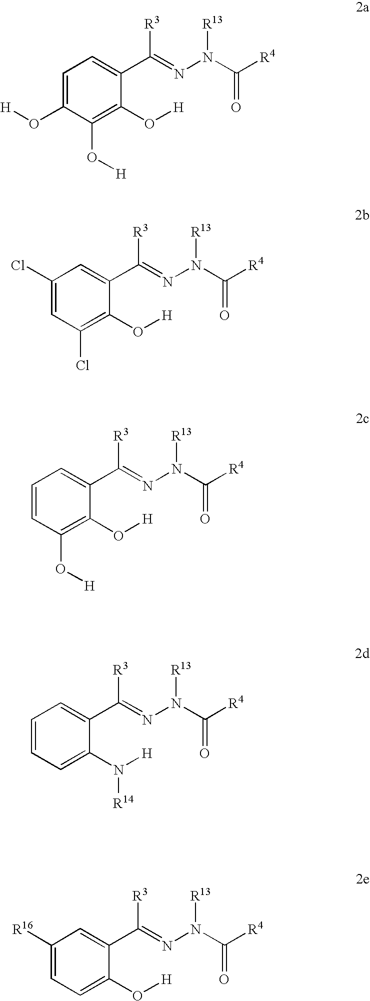
wherein R³, R¹³ and R⁴ have the meaning given in formula 1 and R¹⁴ is hydrogen, lower alkyl , formyl or acetyl and R¹⁶ is hydrogen, methyl, fluoro, chloro, hydroxy or ethyl and pharmaceutically acceptable salts thereof.

3. Compounds of the formulae 3a-3e, 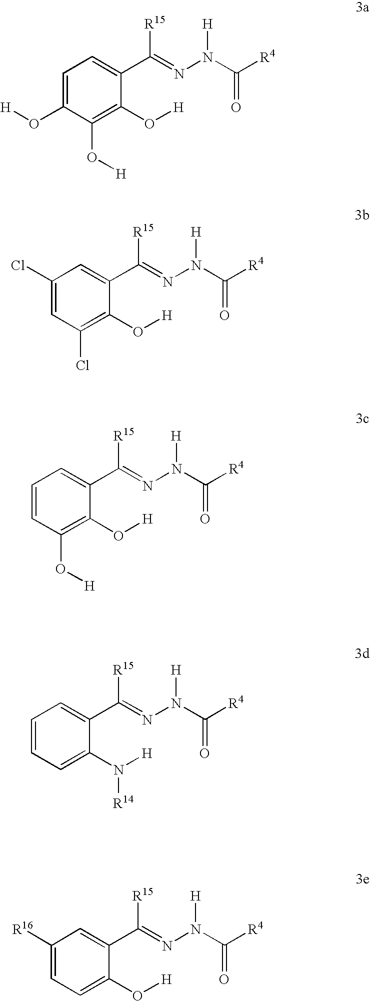
wherein R⁴ has the meaning given in formula 1 and R¹⁴ is hydrogen, lower alkyl formyl or acetyl and R¹⁶ is hydrogen, methyl, fluoro, chloro, hydroxy or ethyl and R¹⁵ is hydrogen, methyl or ethyl and pharmaceutically acceptable salts thereof.

4. Compounds of the formulae 4a-f 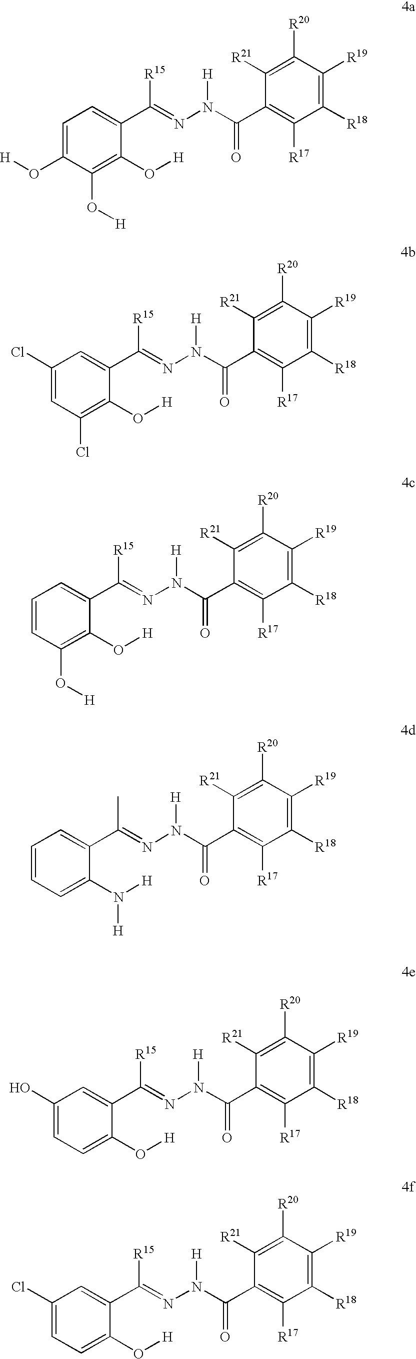
wherein in formula 4a R¹⁵ represents hydrogen, methyl or ethyl and, R¹⁷, R¹⁸, R¹⁹, R²⁰ and, R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R¹⁵ is methyl either one or two of the substituents R¹⁷, R¹⁸, R¹⁹, R²⁰, R²¹ represent N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy or
wherein in formula 4b R¹⁵ represents hydrogen, methyl or ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R¹⁷ is N-pyrrolyl either one or two of the substituents R¹⁸, R¹⁹, R²⁰, R²¹ represent lower alkyl, hydroxy, lower alkoxy, fluoro, chloro, bromo, trifluoromethyl, amino, lower alkylamino, lower alkylendioxy or
wherein in formula 4c R¹⁵ represents hydrogen, methyl or ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R¹⁵ is hydrogen and R¹⁷ is chloro either one or two of the substituents R¹⁸, R¹⁹, R²⁰, R²¹ represents, N-pyrrolyl, 2-pyrrolyl, 3-pyrrolyl, lower alkyl, hydroxy, lower alkoxy, fluoro, chloro, bromo, trifluoromethyl, amino, lower alkylamino or lower alkylendioxy or
wherein in formula 4d R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R¹⁷ is hydrogen or hydroxy, either one or two of the substituents R¹⁸, R¹⁹, R²⁰, R²¹ represent N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy or
wherein in formula 4e R¹⁵ represents hydrogen, methyl, ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy or
wherein in formula 4f R¹⁵ represents hydrogen, methyl, ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, in case R¹⁵ is hydrogen then at least one of the substituents R¹⁷, R¹⁸, R¹⁹, R²⁰ or R²¹ represents pyrrolyl, trifluoromethyl, or lower alkylamino
and pharmaceutically accepable salts thereof.

5. Compounds of the formula 5a-e, 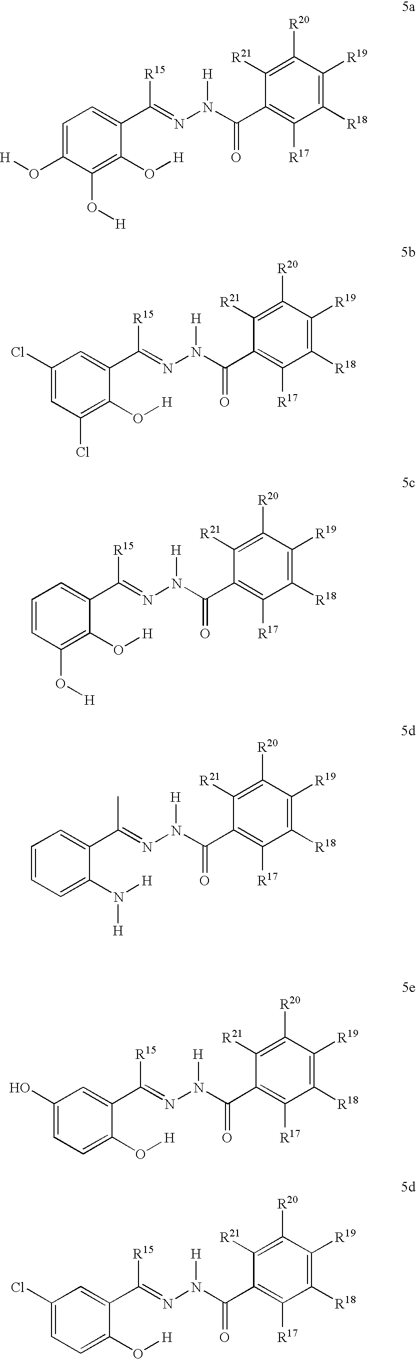
wherein in formula 5a R¹⁵ represents hydrogen, methyl or ethyl and R¹⁷, R¹⁸, R¹⁹, R²⁰ and R²¹, which may be the same or different, represent hydrogen, lower alkyl, hydroxy, lower alkoxy, fluoro, chloro, bromo, trifluoromethyl, lower alkylamino, lower alkylendioxy, with the proviso that one or two of the substituents R¹⁷, R¹⁸, R¹⁹, R²⁰ and R²¹ represent trifluoromethyl or chloro or
wherein in formula 5b R¹⁵ represents hydrogen, methyl or ethyl and R¹⁷, R¹⁸, R¹⁹, R²⁰ and R²¹, which may be the same or different, represent hydrogen, lower alkyl, hydroxy, lower alkoxy, fluoro, chloro, bromo, trifluoromethyl, lower alkylamino, lower alkylendioxy, N-pyrrolyl, 2-pyrrolyl or 3-pyrrolyl, with the proviso that one or two of the substituents R¹⁷, R¹⁸, R¹⁹, R²⁰ and R²¹ represent N-pyrrolyl, 2-pyrrolyl or 3-pyrrolyl, in case R¹⁷ represents N-pyrrolyl, at least one of the substituents R¹⁸, R¹⁹, R²⁰ of R²¹ represents lower alkyl, hydroxy, lower alkoxy, fluoro, chloro, bromo, trifluoromethyl, lower alkylamino, lower alkylendioxy or
wherein in formula 5c R¹⁵ represents hydrogen, methyl or ethyl and R¹⁷, R¹⁸, R¹⁹, R²⁰ and R²¹, which may be the same or different, represent hydrogen, lower alkyl, hydroxy, lower alkoxy, fluoro, chloro, bromo, trifluoromethyl, lower alkylamino, lower alkylendioxy, with the proviso that one or two of the substituents R¹⁷, R¹⁸, R¹⁹, R²⁰ and R²¹ represent chloro or trifluoromethyl or
wherein in formula 5d R¹⁷, R¹⁸, R¹⁹, R²⁰ and R²¹, which may be the same or different, represent hydrogen, lower alkyl, lower alkoxy, fluoro, chloro, bromo, trifluoromethyl, amino, lower alkylamino, lower alkylendioxy, with the proviso that one or two of the substituents R¹⁷, R¹⁸, R¹⁹, R²⁰ and R²¹ represent chloro, methoxy, methyl or trifluoromethyl or
wherein in formula 5e R¹⁵ represents hydrogen, methyl, ethyl and R¹⁷, R¹⁸, R¹⁹, R²⁰ and R²¹ which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, with the proviso that one or two of the substituents R¹⁷, R¹⁸, R¹⁹, R²⁰ and R²¹ represent chloro, methoxy, methyl of trifluoromethyl or
wherein in formula 5f R¹⁵ represents hydrogen, methyl, ethyl and R¹⁷, R¹⁸, R¹⁹, R²⁰ and R²¹, which may be the same or different, represent hydrogen, N-pyrrolyl, 2-pyrrolyl, 3-pyrrolyl, lower alkyl, hydroxy, lower alkoxy, fluoro, chloro, bromo, trifluoromethyl, amino, lower alkylamino, lower alkylendioxy, with the proviso that in case R¹⁵ is hydrogen at least one of the substituents R¹⁷, R¹⁸, R¹⁹, R²⁰ and R²¹ represents N-pyrroly, 2-pyrrolyl, 3-pyrrolyl, trifluoromethyl or lower alkylamino
and pharmaceutically acceptable salts thereof.

6. The end products as described in Examples 1 to 53 and pharmaceutically acceptable salts thereof.

7. Compounds as claimed in claims 1 to 6
N′-(2,5-Dihydroxy-benzylidene)-benzohydrazide
N′-(2-Hydroxy-benzylidene)-2-(1H-indol-3-yl)-acetohydrazide
N′-(2,5-Dihydroxy-benzylidene)-naphthalene-1-carbohydrazide
3,4,5-Trimethoxy-N′-(2,3,4-trihydroxy-benzylidene)-benzohydrazide
2-Amino-5-chloro-N′-(2-hydroxy-benzylidene)-benzohydrazide
3-Trifluoromethyl-N′-(2,4-dihydroxy-benzylidene)-benzohydrazide
3-methoxy-N′-[1-(2-hydroxy-phenyl)-ethylidene]-benzohydrazide
3-Methoxy-N′-(2,5-dihydroxy-benzylidene)-benzohydrazide
3,4-Dichloro-N′-(2,3,4-trihydroxy-benzylidene)-benzohydrazide
4-Chloro-N′-(2,5-dihydroxy-benzylidene)-benzohydrazide
4-Hydroxy-N′-(2,5-dihydroxy-benzylidene)-benzohydrazide
3,4-Dichloro-N′-(2,5-dihydroxy-benzylidene)-benzohydrazide
3-Chloro-N′-(2,5-dihydroxy-benzylidene)-benzohydrazide
4-Hydroxy-3-methoxy-N′-(5-chloro-2-hydroxy-benzylidene)-benzohydrazide
N′-[1-(2,5-Dihydroxy-phenyl)-ethylidene]-benzohydrazide
N′-(2,5-Dihydroxy-benzylidene)-4-hydroxy-3-methoxy-benzohydrazide
N′-(2-Hydroxy-5-methyl-benzylidene)-benzohydrazide
2-Methylamino-N′-(5-chloro-2-hydroxy-benzylidene)-benzohydrazide
2-Methylamino-N′-(2,5-dihydroxy-benzylidene)-benzohydrazide
3-Methyl-N′-(5-chloro-2-hydroxy-benzylidene)-benzohydrazide
3-Trifluoromethyl-N′-(5-chloro-2-hydroxy-benzylidene)-benzohydrazide
2-Methylamino-N′-[1-(2-hydroxy-phenyl)-ethylidene]-benzohydrazide
N-[2-[1-(2-Benzoyl-hydrazono)-ethyl]-phenyl]-acetamide
4-Chloro-N′-[1-(2-amino-phenyl)-ethylidene]-benzohydrazide
3-Methoxy-N′-[1-(2-amino-phenyl)-ethylidene]-benzohydrazide
N′-(2,3-Dihydroxy-benzylidene)-benzohydrazide
3-Methoxy-N′-(2-Hydroxy-benzylidene)-benzohydrazide
N′-(2,3,4-Trihydroxy-benzylidene)-benzohydrazide
N′-(2,4,5-Trihydroxy-benzylidene)-benzohydrazide
3,4,5-Trimethoxy-N′-(2,4,5-trihydroxy-benzylidene)-benzohydrazide
4-Bromo-N′-(2-hydroxy-benzylidene)-benzohydrazide
3-Trifluoromethyl-N′-(2-hydroxy-benzylidene)-benzohydrazide
3-Methyl-N′-(2,5-dihydroxy-benzylidene)-benzohydrazide
3-Trifluoromethyl-N′-(2,5-dihydroxy-benzylidene)-benzohydrazide
4-Hydroxy-N′-[1-(2,5-dihydroxy-phenyl)-ethylidene]-benzohydrazide
4-chloro-N′-(2-hydroxy-3-chloro-benzylidene)-benzohydrazide
4-Chloro-N′-(2,4-dihydroxy-benzylidene)-benzohydrazide
3-chloro-N′-(2-hydroxy-5-chloro-benzylidene)-benzohydrazide
4-Methoxy-N′-(2,3,4-trihydroxy-benzylidene)-benzohydrazide
3,4-Dichloro-N′-(2,3-dihydroxy-benzylidene)-benzohydrazide
3,5-Bis-(trifluoromethyl)-N′-(2,3,4-trihydroxy-benzylidene)-benzohydrazide
3-Chloro-2-pyrrol-1-yl-N′-(2,3,4-trihydroxy-benzylidene)-benzohydrazide
3-Chloro-2-pyrrol-1-yl-N′-(2-hydroxy-3,5-dichloro-benzylidene)-benzohydrazide
2-Pyrrol-1-yl-N′-(2,4,5-trihydroxy-benzylidene)-benzohydrazide
4-Chloro-3-trifluoromethyl-N′-(2,3,4-trihydroxy-benzylidene)-benzohydrazide
4-Chloro-3-trifluoromethyl-N′-(2-hydroxy-3,5-dichloro-benzylidene)-benzohydrazide
4-Chloro-N′-(2,4,5-trihydroxy-benzylidene)-benzohydrazide
N′-(2-Hydroxy-3,5-dichloro-benzylidene)-benzohydrazide
3-Chloro-N′-(2,3,4-trihydroxy-benzylidene)-benzohydrazide
3-Trifluoromethyl-N′-(2,4,5-trihydroxy-benzylidene)-benzohydrazide
3-Trifluoromethyl-N′-(2,3,4-trihydroxy-benzylidene)-benzohydrazide
3,4-Dichloro-N′-[1-(2,3,4-dihydroxy-phenyl)-ethylidene]-benzohydrazide
3,4-Dichloro-N-methyl-N′-(2,3,4-trihydroxy-benzylidene)-benzohydrazide

8. Pharmaceutical compositions for the treatment of infections, containing a compound of any one of claims 1 to 7 and usual carrier materials and adjuvants.

9. Pharmaceutical compositions for the treatment of infections caused by Gram positive and Gram negative pathogens, containing a compound of any one of claims 1 to 7 and usual carrier materials and adjuvants.

10. The compounds of any one of the claims 1 to 7 for use as medicaments for the treatment of infections.

11. The compounds of any one of the claims 1 to 7 for use as medicaments for the treatment of infections caused by Gram positive and Gram negative pathogens.

12. The use of one or more compounds of any one of claims 1 to 7 as active ingredients for the production of pharmaceutical compositions for the treatment of infections.

13. The use of one or more compounds of any one of claims 1 to 7 as active ingredients for the production of pharmaceutical compositions for the treatement of infections caused by Gram positive and Gram negative pathogens.

14. A process for the manufacture of pharmaceutical compositions for the treatment of infections containing one or more compounds as claimed in any one of claims 1 to 7 as active ingredients which process comprises mixing one or more active ingredient with pharmaceutically acceptable excipients in a manner known per se.

15. A process for the manufacture of pharmaceutical compositions for the treatment of infections caused by Gram positive and Gram negative pathogens containing one or more compounds as claimed in any one of claims 1 to 7 as active ingredients which process comprises mixing one or more active ingredient with pharmaceutically acceptable excipients in a manner known per se.
